(19) United States
(12) Patent Application Publication
(10) Pub. No.: US 2024/0141368 A1
(43) Pub. Date: May 2, 2024

(54) METHODS AND COMPOSITIONS FOR IN VIVO DIRECT GENOME EDITING IN PLANTS

(71) Applicants: University of Rhode Island Board of Trustees, Kingstown, RI (US); The Regents of the University of California, Oakland, CA (US)

(72) Inventors: Albert P. Kausch, Charlestown, RI (US); Markita Landry, Berkeley, CA (US)

(21) Appl. No.: 18/495,731

(22) Filed: Oct. 26, 2023

Related U.S. Application Data
(60) Provisional application No. 63/419,576, filed on Oct. 26, 2022.

(51) Int. Cl. C12N 15/82; C12N 9/22

(52) U.S. Cl. CPC C12N 15/8229; C12N 9/22; C12N 2310/20

(57) ABSTRACT

Described herein are methods and compositions for direct genome editing in vivo in plants by targeting cells in the shoot apical meristem.

### RELATED APPLICATION

This application claims the benefit of U.S. Provisional Application No. 63/419,576, filed on Oct. 26, 2022. The entire teachings of the above application are incorporated herein by reference.

### GOVERNMENT SUPPORT

This invention was made with government support under grant number DE-SC0018277 awarded by the US Department of Energy and grant number 1444478 awarded by the National Science Foundation. The government has certain rights in the invention.

### STATEMENT REGARDING SEQUENCE LISTING

The Sequence Listing associated with this application is provided in .xml format in lieu of a paper copy and is hereby incorporated by reference into the specification. The name of the .xml file containing the Sequence Listing is UNRI-001 101_SEQ_LISTING.xml. The xml file is 12.0 kilobytes and was created on Oct. 26, 2023.

### BACKGROUND OF THE INVENTION

Plant transformation involves the ability to insert and stably integrate DNA constructs into plant genomes. In comparison, CRISPR mediated genome editing allows for single base pair insertions, deletions, and base editing to achieve stable genetic outcomes. Standard integrative plant transformation is a major bottleneck technology to enable CRISPR mediated genome editing in plants. Current methods for plant genome editing require standard plant transformation processes to deliver the CRISPR reagents, and the edited cells need to be regenerated through tissue culture to whole plants containing the edit. There is a need for direct genome editing in plants, uncoupling the standard transformation process to genome editing.

### SUMMARY OF THE INVENTION

In one aspect, provided herein is a method of genome editing in an explant, comprising: obtaining a seed of a bridge transgenic plant having a stably integrated Cas expression cassette containing a nucleic acid encoding a Cas protein; treating the seed to obtain a germinated seed, wherein the germinated seed contains a viable epicotyl and the Cas protein is expressed in the epicotyl; and carrying out genome editing in the germinated seed.

In some embodiments, the method further comprises: prior to the genome editing step, cutting the germinated seed to produce a seed section in which a portion of shoot apical meristem is exposed or proximate to an exposed surface of the seed section; and applying a genome editing reagent directly in vivo to the portion of shoot apical meristem or the exposed surface of the seed section under conditions allowing delivery of the genome editing reagent into a cell of the shoot apical meristem, wherein the cell expresses the Cas protein, whereby genome editing occurs in the cell to produce at least one genomic edit.

In some embodiments, the bridge transgenic plant is a monocotyledonous, dicotyledonous, monoploid, diploid, triploid, or polyploid plant.

In some embodiments, the seed section is produced by cutting the germinated seed longitudinally. In some embodiments, the seed section is produced by isolating a section of the germinated seed containing the shoot apical meristem.

In some embodiments, applying the genome editing reagent to the exposed portion of shoot apical meristem or the exposed surface of the seed section includes substantially covering the portion of shoot apical meristem or the exposed surface with a composition containing the genome editing reagent.

In some embodiments, the seed section is subjected to vacuum filtration or microprojectile bombardment or both. In some embodiments, the seed section is subjected to one or both of a cold pretreatment and osmotic pretreatment prior to, before, or at the same time with applying the genome editing reagent.

In some embodiments, the genome editing reagent includes a guide RNA (gRNA) or a vector for expressing the gRNA.

The genome editing reagent can include two or more gRNAs for making at least two different genome edits, whereby genome editing of at least two loci occurs in the cell of the shoot apical meristem, and wherein at least one of the genome edits confers a selective advantage under a selective agent or condition. The selective advantage can be resistance or tolerance to a condition or toxic agent. In some embodiments, the selective agent or condition is applied to the seed section before, after, or at the same time that the genome editing reagent is applied, whereby conferring the selective advantage to the cell containing the at least two genome edits.

In some embodiments, the genome editing reagent includes a vector or delivery vehicle carrying a nucleic acid, e.g., a vector for expressing the gRNA or a delivery vehicle complexed with the gRNA. In some embodiments, the delivery vehicle is a carbon nanotube (e.g., a carbon nanotube functionalized to bind a nucleic acid). In some embodiments, the vector is a viral vector (e.g., a Foxtail mosaic virus vector).

In another aspect, described herein is a method of producing a genome-edited plant, comprising: obtaining a seed of a bridge transgenic plant having a stably integrated Cas expression cassette containing a nucleic acid encoding a Cas protein; treating the seed to obtain a germinated seed, wherein the germinated seed contains a viable epicotyl and the Cas protein is expressed in the epicotyl; sterilizing the germinated seed; cutting the sterilized seed to produce a seed section in which a portion of shoot apical meristem is present and exposed or proximate to an exposed surface; applying a genome editing reagent directly in vivo to the exposed surface or the portion of shoot apical meristem of the seed section under conditions allowing delivery of the genome editing reagent into a cell of the shoot apical meristem, wherein the cell expresses the Cas protein, whereby genome editing occurs in the cell to produce at least one genome edit; growing the seed section under conditions allowing whole plant regeneration, whereby an F1 genome-edited plant is produced.

In some embodiments, the bridge transgenic plant used in the method for producing a genome-edited plant is a monocotyledonous, dicotyledonous, monoploid, diploid, triploid, or polyploid plant.

In some embodiments, the seed section is produced by cutting the germinated seed longitudinally. In some embodiments, the seed section is produced by isolating a section of the germinated seed containing the shoot apical meristem.

In some embodiments, applying the genome editing reagent to the exposed portion of shoot apical meristem or the exposed surface of the seed section includes substantially covering the portion of shoot apical meristem or the exposed surface with a composition containing the genome editing reagent.

In some embodiments, the seed section is subjected to vacuum filtration or microprojectile bombardment or both. In some embodiments, the seed section is subjected to one or both of a cold pretreatment and osmotic pretreatment prior to, before, or at the same time with applying the genome editing reagent.

In some embodiments, the genome editing reagent includes a gRNA or a vector for expressing the gRNA.

In some embodiments, the genome editing reagent includes a vector or delivery vehicle carrying a nucleic acid, e.g., a vector for expressing a gRNA or a delivery vehicle complexed with the gRNA. In some embodiments, the delivery vehicle is a carbon nanotube (e.g., a carbon nanotube functionalized to bind a nucleic acid). In some embodiments, the vector is a viral vector (e.g., a Foxtail mosaic virus vector).

In some embodiments, the genome editing reagent includes two or more gRNAs or vectors for expressing the gRNAs for making at least two different genome edits, whereby genome editing of at least two loci occurs in the cell of the shoot apical meristem, and wherein at least one of the genome edits confers a selective advantage under a selective agent or condition and at least one of the genome edits confers a target trait, and wherein the F1 genome-edited plant contains both genome edits. In some embodiments, the selective advantage is resistance or tolerance to a condition or toxic agent.

In some embodiments, the method for producing a genome-edited plant further comprises applying the selective agent or condition to the seed section before, after, or at the same time that the genome editing reagent is applied, or before or during the growing step, whereby conferring the selective advantage to the cell containing the at least two genome edits.

The method for producing the genome-edited plant can further comprise producing from the F1 genome-edited plant a hybrid embryo, seed or plant containing the genome edit conferring the target trait in which the Cas expression cassette is removed by segregation in subsequent generations.

The hybrid embryo, seed or plant can be an F1BC1 or F1BC2 hybrid embryo, seed or plant produced by backcrossing the F1 genome-edited plant to a plant germplasm having the same or closely syntenic genotype as the F1 genome-edited plant but lacking the Cas expression cassette and the at least one genome edit. In some embodiments, an offspring containing the genome edit conferring the target trait is segregated away from the Cas expression cassette to produce a non-GMO, genome-edited intact fertile plant.

In some embodiments, the method further comprises producing from the F1 genome-edited plant a hybrid embryo, seed or plant containing the genome edit conferring the target trait in which the Cas expression cassette and the genome edit conferring the selective advantage are both removed by segregation in subsequent generations. The hybrid embryo, seed or plant can be an F1BC1 or F1BC2 hybrid embryo, seed or plant produced by backcrossing the F1 genome-edited plant to a plant germplasm having the same or closely syntenic genotype as the F1 genome-edited plant but lacking the Cas expression cassette and the at least one genome edit. An offspring containing the genome edit conferring the target trait can be segregated away from the Cas expression cassette and the genome edit conferring the selective advantage to produce a non-GMO, genome-edited intact fertile plant.

In some embodiments, the method further comprises using the F1 genome-edited plant in a wide cross to a plant germplasm having a distantly syntenic genotype as the F1 genome-edited plant lacking the Cas expression cassette and the genome edit conferring the selective advantage to produce a bridge intermediate F1BC1 hybrid embryo, seed, or plant. In some embodiments, an offspring containing the genome edit conferring the target trait is segregated away from the Cas expression cassette and the genome edit conferring the selective advantage to produce a non-GMO, genome-edited intact fertile plant.

In some embodiments, an F2 hybrid embryo, seed or plant is recovered to produce a fertile embryo, seed or hybrid plant using in situ embryo rescue.

In another aspect, provided herein is a genome-edited seed section or epicotyl section, comprising a surface including an exposed portion of shoot apical meristem, wherein cells in the shoot apical meristem contain a stably integrated Cas expression cassette containing a nucleic acid encoding a Cas protein and an edited genome. In some embodiments, the edited genome contains at least one genome edit that confers a target trait. In some embodiments, the edited genome further contains a genome edit that confers a selective advantage under a selective agent or condition.

### BRIEF DESCRIPTION OF THE DRAWINGS

FIG. 1 is a scheme illustrating creation of a stable line with editing machinery that can be backcrossed with initial variety to create edits directly in a commercial line.

FIG. 2 is a scheme illustrating creation of a stable line with editing machinery using a guide RNA to convert an existing sequence to a selectable marker where co-editing via multiplexing occurs and can be co-inherited in a closely syntenic variety.

FIG. 3 is a scheme illustrating use of multiplexing to co-edit additional genes and in a breeding platform to include two parents of closely related germplasms.

FIG. 4 is a scheme illustrating creation of a stable parental line with editing machinery that can be crossed with incompatible lines to create edits in wide crosses.

FIG. 5 is a scheme illustrating use of a wide cross bridge intermediate in an in situ embryo rescue to recover the stable wide cross with edits.

FIG. 6 is a set of schemes showing various Cas9 expression constructs for creating transgenic plants.

FIG. 7 is a bar graph showing significant reduction in GFP florescence in leaves of plant regenerated from seed sections treated with gRNAs carried by singled-walled carbon nanotubes compared with the positive controls (pos-1, -2, -3, and -4) and calibrated against negative wild-type controls (neg-1 and -2).

### DETAILED DESCRIPTION OF THE INVENTION

This disclosure relates to methods, systems and compositions for direct in vivo genome editing in plants. As described herein, a platform technology or system for direct genome editing is developed utilizing developing epicotyls. This platform includes a stable transgenic line with an integrated gene expressing an editing protein, such as a Cas polypeptide (e.g., Cas9). The editing gene is driven by a promoter and expressed in the stem cell initials in the shoot apical meristem (SAM) of the transgenic line. The epicotyls of these lines are used as targets of various direct editing reagent delivery approaches, including but not limited to carbon nanotubes (CNTs), non-integrating plant virus delivery systems, e.g., Foxtail Mosaic Virus (FoMV) or Barley Stripe Mosaic Virus (BSMV), protein nanoparticles, polycations, liposomes, silica nanoparticles and ribonucleoproteins (RNPs). Using a line containing a stably integrated and expressed genome editing gene and an appropriate reagent delivery approach, reagents for genome editing, such as guide RNAs (gRNAs), are directly contacted with the stem cell initials in the shoot apical meristem, facilitating in vivo editing in plants.

It is desirable to edit the stem cell initials in the shoot apical meristem, also known as the organizing center (OC), as these are known to fate map as the germline initials involved in egg and sperm seed development in the flower. These stem cell initials in the SAM are the target cells of interest for direct genome editing in this disclosure. These target cells are interacted with editing reagents, such as components of a CRISPR/Cas system, using various delivery methods, such as carbon nanotubes, or non-integrating virus. The treated epicotyls with edited cells can be grown in regeneration media to a whole fertile plant with edited cells.

### Definitions

As used herein, a “plant” is any plant, particularly an agronomically useful plant (e.g., seed plant). A “plant cell” is a structural and physiological unit of the plant, which comprises a cell wall but may also refer to a protoplast. The plant cell may be an isolated single cell or a cultured cell, or as a part of higher organized units such as for example, a plant tissue, or a plant organ differentiated into a structure that is present at any stage of a plant's development. A plant may be a monocotyledonous or dicotyledonous plant species.

The term “bridge intermediate” as used herein refers to a genetic bridge (e.g., a transgenic plant) for importing one or more genes into a hybrid, providing a mechanism for importing any new genes not found in common breeding program materials and any de novo genetic material that arises from these wide varietal, species or genera crosses using traditional plant breeding techniques.

As used herein, the term “desirable trait”, “desired trait”, or “target trait” refers to a characteristic which may be beneficial or advantageous, such as a commercially valuable or agronomically important trait, or a useful trait for research purposes. Examples include, but are not limited to: resistance to insects, other pests, or disease-causing agents (such as viral, bacterial, fungal, and nematode agents); tolerance or resistance to herbicides; enhanced stability; increased yield or shelf-life; environmental tolerances (such as tolerance to drought, heat, chilling, freezing, excessive moisture, salt stress, or oxidative stress); male sterility; size (e.g., larger grain size); and nutritional enhancements (such as starch quantity and quality; oil quantity and quality; protein quality and quantity; vitamin quality and quantity; amino acid composition; and the like).

An “egg” in a seed plant is an ovum (plural; ova). It is a haploid female reproductive cell or gamete.

An “asexual plant” is a plant lacking floral structures such that it is incapable of participating either as a male or female parent in sexual reproduction and propagates vegetatively.

As used herein, a “plant part” refers a part of a plant, including single cells and cell tissues such as plant cells that are intact in plants, cell clumps and tissue cultures from which plants can be regenerated. Plant parts also include explants.

As used herein, the term “explant” refers to an organ or tissue, part of a tissue or tissues, or pieces of tissues or organs derived from a plant or a plant part, such as a germinating seed. An explant can be a part of a plant, such as an epicotyl, a leaf, an immature embryo, etc. or can be derived from an organ as in a part of the shoot, seed or any other tissue of a plant or seed.

The term “hybrid plant” refers to a plant produced by crossing two parents of different genotypes or germplasm backgrounds.

The term “in planta” refers to a method or a process performed on a plant or tissue without involving an in vitro method or process of cultivating plant tissues or organs. For example, in planta transformation refers to a transformation method performed on a plant or tissue without any tissue culturing steps performed on any excised tissues or organs. For clarity, tissue culturing steps do not include growing a plant on or in growth media, hydroponics, media plates, etc.

“Plant breeding” is application of genetic analysis to development of plant lines better suited for human purposes.

As used herein, a “progeny” is the descendant of a particular cross. The descendant can be, for example, of the F1, the F2, or any subsequent generation or as TO, T1, T2 as describing generations in transgenic plants.

The terms “Cas polypeptide” and “Cas protein” can be used herein interchangeably. Exemplary Cas proteins include Cas3, Cas4, Cas9, Cas10, TaCas9, and dCas proteins.

The term “guide nucleic acid” as used herein refers to a nucleic acid that recognizes or hybridizes to another nucleic acid in a sequence-specific manner. In some embodiments, the guide nucleic acid is a guide RNA (gRNA). A gRNA can be a single polynucleotide (sgRNA) or a duplex of two polynucleotides. The term “gRNA” is inclusive of both gRNA structures.

The term “CRISPR/Cas system” as used herein refers to a nucleic acid-targeting system that utilizes at least one Cas polypeptide and at least one guide nucleic acid to target a specific nucleic acid sequence (e.g., a genomic sequence or site in a cell).

The term “genome editing reagent” as used herein refers to a reagent, component, or molecule that can, working alone or with other reagents, components, or molecules, induces or creates a genomic edit such as a deletion, insertion, indel, substitution or other modification in the genome of a cell, e.g., by creating a single or double-stranded break in the genome and allowing homologous recombination to modify a genomic site. Examples of genome editing reagents include components of CRISPR/Cas systems (e.g., Cas proteins, gRNAs, sgRNAs, and homologous recombination templates), zinc finger nucleases and Transcription Activator-Like Effector (TALE) Nucleases. A genome editing reagent can include a nucleic acid (e.g., a gRNA) or protein complexed to or carried by a carrier or vehicle such as a vector, virus, nanoparticle, polycation, liposome, or carbon nanotube. In some embodiments, a genome editing reagent includes a vector for expressing a nucleic acid (e.g., gRNA) or a protein.

The term “genetically modified organism” or “GMO” in reference to a plant as used herein is a transgenic plant, e.g., one containing one or more transgenes that have been artificially inserted.

The term “wild type” refers to a reference, which can be an organism, cell, set of genes, gene, or nucleotide sequence. For purposes herein, wild type refers to a parent of a hybrid progeny.

A first nucleic acid sequence is “operably linked” to a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

The terms “regulatory sequence” and “promoter” are used interchangeably herein, and refer to nucleic acid sequences, such as initiation signals, enhancers, and promoters, which induce or control transcription of protein coding sequences with which they are operatively linked. In some examples, transcription of a recombinant gene is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally-occurring form of a protein. In some instances, the promoter sequence is recognized by the synthetic machinery of the cell, or introduced synthetic machinery, required for initiating transcription of a specific gene. A promoter may be constitutive or inducible. Specific, non-limiting examples of promoters that can be used to practice the disclosed methods include, but are not limited to, floral-specific promoters, constitutive promoters, as well as inducible promoters such as heat shock promoters, glucocorticoid promoters, and chemically inducible promoters. Promoters produced by recombinant DNA or synthetic techniques may also be used. Useful promoters known in the art include Act1, Adh1, Ubi-1, Gmubi, CaMV35S, nos, KST1, TCTP, PR-la, GST1, SGD24-STR246C, Zmap, GCC, and GSSP1 promoters.

The term “selectable marker” refers to a nucleic acid sequence or protein that confers a selectable phenotype, such as in plant cells, that facilitates identification of cells containing a nucleic acid sequence. Transgenic plants expressing a selectable marker can be screened for transmission of the gene(s) of interest. Examples of selectable markers include but are not limited to genes that confer resistance to toxic chemicals (e.g., ampicillin, spectinomycin, streptomycin, kanamycin, geneticin, hygromycin, glyphosate or tetracycline resistance, as well as bar and pat genes which confer herbicide resistance), complement a nutritional deficiency (e.g., uracil, histidine, or leucine), or impart a visually distinguishing characteristic (e.g., color changes or fluorescence, such as 13-gal or green fluorescent protein).

The term “syntenic genotype” refers to two or more genes residing on the same chromosome of a given genotype even if genetic linkage cannot be demonstrated or tested for. Two species have the same syntenic genotype if they have the same co-localization of the two or more genes.

The term “vector” refers to a carrier DNA molecule into which a DNA sequence can be inserted for introduction into a host cell. Preferred vectors are those capable of autonomous replication and/or expression of nucleic acids to which they are linked. Vectors capable of directing the expression of genes to which they are operatively linked are referred to herein as “expression vectors”. Thus, an “expression vector” is a specialized vector that contains the necessary regulatory regions needed for expression of a gene of interest in a host cell. In some embodiments, the gene of interest is operably linked to another sequence in the vector. Vectors can be viral vectors or non-viral vectors.

A virus or vector “transduces” or “transfects” a cell when it transfers nucleic acid into the cell. A cell is “transformed” by a nucleic acid transduced into the cell when the DNA becomes stably replicated by the cell, either by incorporation of the nucleic acid into the cellular genome, or by episomal replication. As used herein, the term “transformation” encompasses all techniques by which a nucleic acid molecule can be introduced into such a cell. Examples include, but are not limited to, transfection with viral vectors, transformation with plasmid vectors, electroporation, lipofection, Agrobacterium-mediated transfer, direct DNA uptake, microneedle injection, and microprojectile bombardment.

Transgenic Plant with Stably Integrated Gene for Mediating Genome Editing

In one aspect, provided herein is a transgenic plant containing a stably integrated gene expressing a protein for mediating genome editing (e.g., a Cas protein, Cas9, or TaCas9). In some embodiments, the transgenic plant does not contain the desired genome edit or all of the genome editing reagents required for genome editing (e.g., gRNAs). Rather, it serves as a bridge intermediate for editing genes in elite backgrounds without a tissue culture intermediate as required in standard protocols.

The transgenic line can be generated using an expression cassette and standard integrative transformation technologies, such as Agrobacterium-mediated transformation of immature embryo explants, selection for the integration event, and plant regeneration to fertility. See, Nelson-Vasilchik et al., In Vitro Cell.Dev.Biol.-Plant 58, 331-342 (2022); Kausch et al., Maize tissue culture, transformation, and genome editing. Ed. David Songstad, In Vitro Cell & Devel Biol Plant 2021. Plant Springer publ.; and Kausch et al., 2021, Maize Transformation: History, Progress and Perspectives, Molecular Breeding 41, 38, Springer publication. The TO plants are selfed to generate segregating T1 plants which can be screened by a selectable marker. The transgenic plants can be analyzed and confirmed by PCR, Southern blots, or Q-PCR.

The expression cassette or construct (e.g., a Cas cassette) can include a gene for mediating genome editing (e.g., a gene encoding a Cas protein) operably linked to a promoter (e.g., a constitutive or inducible promoter) suitable for the plant. The expression cassette can further include one or more selectable markers (e.g., BAR). In some embodiments, the expression cassette can also contain a GFP gene operably linked to the gene for mediating genome editing. In later steps, GFP expression can be used as a visual marker of, for example, Cas polypeptide (e.g., Cas9) expression in cells.

The transgenic bridge intermediate can be generated in any elite variety using homology directed repair (HDR)-based methods and used as an editing platform in an elite background. Then in one subsequent generation, the Cas cassette in the transgenic bridge intermediate can be crossed out from the elite background, resulting in a non-GMO edited variety.

A monocotyledonous or dicotyledonous plant species can be used to generate a transgenic bridge intermediate. The plant can be a monoploid, diploid, triploid, or polyploid plant. Exemplary plants include but are not limited to cereal species such as, sorghum, maize, rice, wheat and barley; vegetable species such as tomato, lettuce, pepper, cucumber, garlic, and carrots; commodity crops such as canola, cotton, soybean, tobacco, cannabis, pennycress, coffee, tea, sugarcane, and sugar beet; fruit and nut species such as citrus, apple, melons, strawberry, almonds, raspberry; and horticultural species such as Petunia, lilies, orchids and roses.

### Targeting Cells of Shoot Apical Meristem

In the methods and systems described herein, genome editing occurs in the cells in the shoot apical meristem in a transgenic bridge intermediate. Thus, the cells in the shoot apical meristem (SAM) are contacted with one or more genome editing reagents (e.g., gRNA) such that the reagents can be delivered into these cells.

To facilitate contact between the cells in the SAM and the genome editing reagent, a part of the transgenic bridge intermediate containing a viable epicotyl or the SAM can be cut or isolated such that the cells in the SAM are exposed or proximate to an exposed surface.

In some embodiments, a seed of the transgenic bridge intermediate can be germinated to obtain a germinated seed containing a viable epicotyl. Preferably, a germinated seed is selected when the radicle has just emerged and before or just after the emergence of the epicotyl. In some embodiments, the seed is germinated for about 10 to 24 hours. The seed can be germinated using techniques known in the art, e.g., in an appropriate media or water. Examples of media include Kausch_sorghum_rgn2 Medium, Murashige and Skoog Basal Medium (0.5X), Schenck and Hildebrandt Medium, and Gamborg's Basal Medium. See, e.g., Nelson-Vasilchik et al., In Vitro Cell.Dev.Biol.-Plant 58, 331-342 (2022); Murashige et al., Physiologia Plantarum. 15 (3): 473-497 (1962); Schenk et al., Canadian Journal of Botany, 50, 199-204 (1972); and Gamborg et al., Experimental Cell Research, 50, 151-158 (1968).

Germination is not synchronous in a population of seeds, so development and rate of growth may vary between seeds. A skilled practitioner would be able to monitor a population of germinating seeds to select appropriate ones based on the guidance provided herein.

In some embodiments, to expose the cells in the SAM, a selected germinated seed can be first surface sterilized (e.g., using a procedure described herein or known in the art) and then dissected or cut, preferably, about in half longitudinally (e.g., bisected) approximately through the center of the epicotyl, creating two separate seed sections each with an exposed surface exposing target cells in the SAM. In some embodiments, the target cells in the SAM can be right under an exposed surface (e.g., a few cells deep). It is contemplated that more than one cut can be made to a seed. The portions of the cut seed need not have the same size or same area of exposed surfaces. In some embodiments, a seed is not cut entirely through (e.g., at least 50% to 95% but less than 100% through longitudinally) such that the seed portions or sections are still connected. In any event, a skilled practitioner would understand that maximizing exposure of the cells in the SAM would be beneficial to the methods described herein.

In some embodiments, thin sections (e.g., about 150 lam, or about 100 to 250 μm thick sections) through the SAM of the epicotyl or the plumule can also be isolated and used. Each section contains exposed cells in the SAM.

Dissection can be accomplished using a curved scalpel blade in a sterile petri dish in a laminar flow hood on a seed placed on sterile filter paper moistens with an appropriate media. Examples of media include Kausch_sorghum_rgn2 Medium, Murashige and Skoog Basal Medium (0.5X), Schenck and Hildebrandt Medium, and Gamborg's Basal Medium. Alternatively, tungsten surgical blades or a Vibratome can be used to generate seed sections.

After dissection, one or more seed portions or sections can be placed onto a solid medium with the cut or exposed surface facing up for the next steps. Alternatively, one or more seed portions or sections can be placed beneath a cover glass in a liquid media. Examples of media include Kausch sorghum_rgn2 Medium, Murashige and Skoog Basal Medium (0.5X), Schenck and Hildebrandt Medium, Gamborg's Basal Medium, and other appropriate media.

### Delivering Genome Editing Reagents to Cells of Shoot Apical Meristem

Exposed cells of the SAM in a part (e.g., a seed portion or section) of a transgenic bridge intermediate can be contacted with an appropriate delivery agent or vector to carry one or more gRNAs or other editing reagents targeting one or more genes of interest. Delivery agents or vectors include nanoparticles and carbon nanotubes carrying the gRNAs. A delivery agent or vector can also be a non-integrating virus carrying a gRNA to edit a gene of interest. In some embodiments, a Cas gene is integrated and expressed in target cells of interest in the SAM (i.e., the stem cell initials in the SAM). In some embodiments, the gRNAs or other editing reagents are delivered naked (e.g., without being associated with any delivery agents, carriers, or vehicles such as viruses, viral or non-viral vectors, proteins, polymers, liposomes, or nanoparticles). When the gRNAs are appropriately presented and delivered to the target cells of interest in the SAM expressing a Cas protein, genome editing can be actuated in situ. The SAM then continues to grow with edited cells. When referring to delivery of a gRNA into a cell, it can be delivering the gRNA itself or, for example, a vector for expressing the gRNA in the cell.

Delivery of one or more genome editing reagents to cells in the SAM can utilize one or a combination of carriers including but not limited to carbon nanotubes (CNTs), non-integrating plant virus delivery systems such as Foxtail Mosaic Virus (FoMV) and Barley Stripe Mosaic Virus (BSMV), protein nanoparticles, polycations, liposomes, silica nanoparticles, and ribonucleoproteins (RNPs). See, Que et al., Front Plant Sci 5:379-379 (2014); Yadava et al., Front Plant Sci 7:1949 (2017); Kausch et al., Plant Sci 281:186-205 (2019); Kausch et al., Maize tissue culture, transformation, and genome editing. Ed. David Songstad, In Vitro Cell & Devel Biol Plant 2021. Plant Springer publ.; and Kausch et al., 2021, Maize Transformation: History, Progress and Perspectives, Molecular Breeding 41,38, Springer publ. Osmotic treatments, cold treatments, microprojectile bombardments, or combinations thereof could also be used to facilitate delivery of genome editing reagents naked or carried by a delivery vehicle.

Carbon nanotubes (CNTs), and other high aspect ratio nanomaterials, have been shown to pass through the plastid envelope in extracted chloroplasts and plant cell membranes. Tracking of cargo-nanoparticle complexes in complex plant tissues can be accomplished using single-walled carbon nanotubes (SWNTs) with strong intrinsic near-infrared (nIR) fluorescence. CNTs are below exclusion limit of the plant cell wall (˜20 nm), offering a significant option as a macromolecular delivery method. Pristine or functionalized carbon nanotubes (e.g., carboxylated carbon nanotubes) can be used to carry and deliver nucleic acids using methods known in the art. See, e.g., US 2020/0063148; Demirer et al., Nature Protocols, 14: 2954-2971 (2019); and Demirer et al., bioRxiv 564427; doi:10.1101/564427 (2019). CNTs and SWNTs are commercially available.

Genome editing reagents such as gRNAs (e.g., sgRNAs) targeting one or more genes of interest can be designed using methods known in the art, considering locations, sequences, efficacies, on-target activities, off-target effects, and other factors. See, Songstad et al., Crit Rev Plant Sci 36:1-23 (2017); Chen et al., Annu Rev Plant Biol 70:667-697 (2019); Metje-Sprink et al., Front Plant Sci 9:1957 (2019); Razzaq et al., Int J Mol Sci 20:4045 (2019). A Cas protein-gRNA complex can target a specific genomic site to allow editing. Generally, a gRNA can form a complex with a Cas protein and also contains a guide or spacer sequence with sufficient complementarity to a target sequence of interest to bind to the sequence. In some embodiments, multiplexed gRNAs for multi-locus editing can be used in the methods described herein. See, Kausch et al. 2019; Metje-Sprink et al. 2019; and Razzaq et al. 2019. For example, multiplexed gRNAs can be designed to edit a gene of interest and another gene that confers a selective advantage under one or more selective conditions. A selective condition can be applied before, after or at the same time the one or more genome editing reagent are applied to the exposed cells of the SAM to preferentially select for genome-edited cells. For example, the selective advantage can be resistance or tolerance to a condition (e.g., cold, heat, nutritional deficit, or water shortage) or toxic chemical.

In some embodiments, a genome editing reagent (e.g., gRNA) is applied to a seed portion or section by directly contacting the seed section with a composition (e.g., a fluid or a solution) containing the genome editing reagent (e.g., by applying the composition directly to an exposed surface of the seed portion or section so the entire surface or most of it is covered by or flooded with the composition, or by immersing or submerging the seed portion or section in the composition). In some embodiments, the method includes subjecting the seed portion or section to vacuum filtration or microprojectile bombardment. In some embodiments, the method further includes subjecting the seed portion or section to one or both of a cold treatment and osmotic treatment prior to, during, or after contacting the seed portion or section with the composition containing the genome editing reagent.

Genome edits in the cells in the SAM can be confirmed using standard techniques such as PCR, Southern blot analysis, RT-PCR, sequencing, florescence microscopy, X-ray microscopy and confocal microscopy.

Within the scope of the invention is a seed section, an epicotyl section, or a cut seed, containing a surface including an exposed portion of the SAM, generated from a seed of a transgenic bridge intermediate as described herein. Cells in the seed section, seed portion or cut seed contain a stably integrated expression cassette containing a Cas gene. In some embodiments, the cells in the SAM in the seed section, seed portion or cut seed contain one or more genomic modifications not in the transgenic bridge intermediate.

### Systems for In Vivo Genome Editing in Plants

The present invention includes systems for in vivo genome editing in plants. Generally, a system of the present invention includes one or more transgenic plants, seeds, plant parts, intermediates, reagents, or compositions for carrying out the methods for in vivo genome editing in plants described herein. For example, a system can include a transgenic plant or transgenic line described herein (e.g., a bridge intermediate), which contains a stably integrated gene for mediating genome editing (e.g., an expressed gene encoding a Cas protein), a seed of the transgenic plant or transgenic plant line, or a genome-edited seed section or epicotyl section produced from the transgenic plant by the methods described herein.

The system can further include one or more additional reagents for editing the genome of the cells in the SAM in seeds of the transgenic plant, e.g., gRNAs, vectors or other materials for making gRNAs selective for the Cas protein expressed in the cells in the SAM, vectors or vehicles designed for carrying gRNAs for delivery into the cells, or vectors or vehicles carrying the gRNAs for delivery into the cells.

### Generating Genome-Edited Plants

Genome-edited seed sections generated from transgenic bridge intermediates using the methods described herein can be grown to produce genome-edited plants. The genome-edited plants can then be backcrossed or outcrossed to remove any transgenes (e.g., expression cassettes containing a Cas gene or selectable marker), facilitating recovery of non-GMO (e.g., transgene-free) hybrids that can be rapidly introduced to the agricultural market.

The platforms, systems and methods described herein provide a breeding strategy which includes using either non-integrative or stable integrative transformation approaches for providing Cas functionality to a sexually compatible germplasm. The construction of a parental line with the integrated editing machinery (e.g., Cas proteins) provides a foundation line for sexually compatible species or varieties, which can serve as a bridge intermediate for direct genome editing and then backcrossed with the parental line or crossed with sexually compatible germplasm. This strategy is useful for mobilizing edited sequences directly into elite germplasm and closely related varieties across a broad range of germplasm required for market penetration. The initial foundation line (e.g., the bridge intermediate expressing a Cas protein) constitutes an “allele factory”, where the editing machinery can be maintained across generations to continue introducing and capitalizing on novel genetic variants.

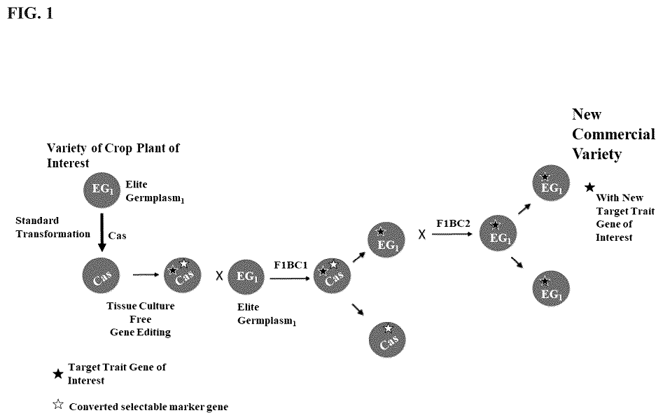

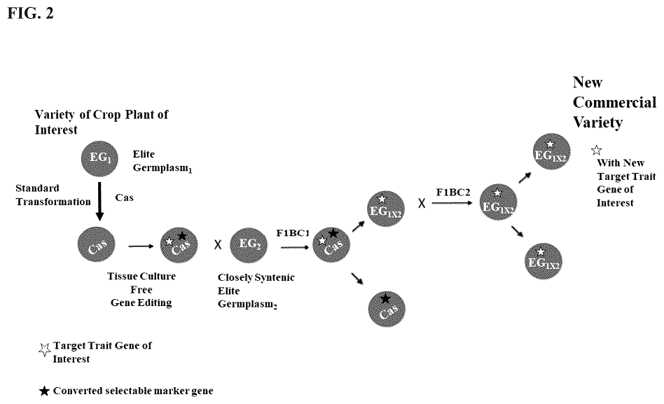

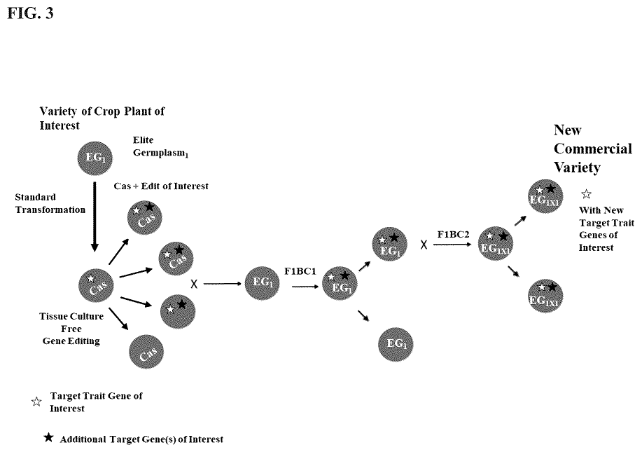

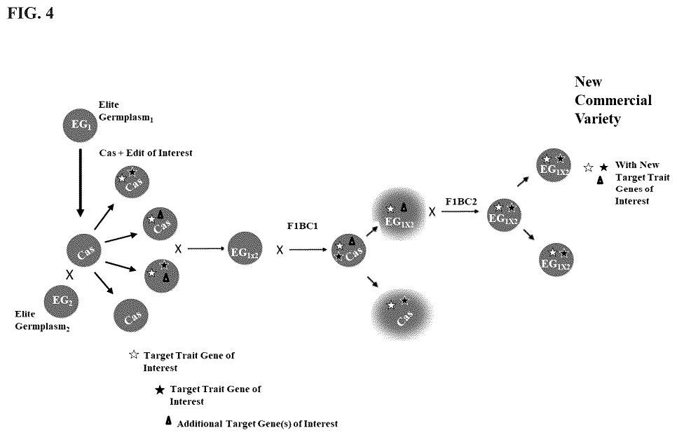

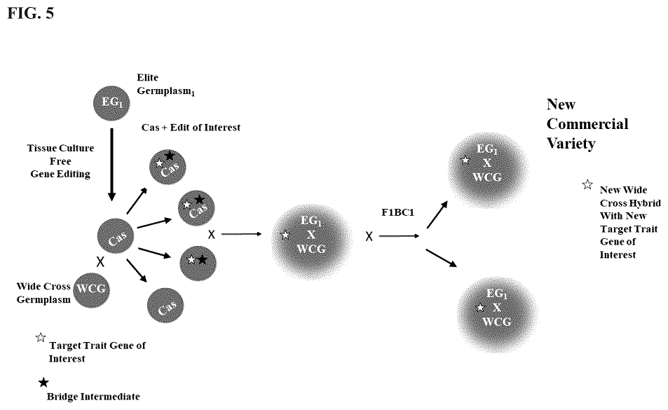

Exemplary processes for generating genome-edited non-GMO plants are illustrated in FIGS. 1-5. Creating a stable line expressing an editing machinery (e.g., a Cas protein) that can be backcrossed with initial variety facilitates genome-editing directly in a commercial line. See FIG. 1. The process can include conversion of a selectable marker gene via multiplex genome-editing. Genotype independent transformation can further expand utility. FIG. 2 illustrates generation of a stable line with editing machinery using a guide RNA to convert an existing sequence to a selectable marker where co-editing via multiplexing occurs and can be co-inherited in a closely syntenic variety. Multiplexing can be used to co-edit additional genes and used in the breeding platform to include two parents of closely related germplasm. See FIG. 3. In addition, a stable parental line expressing an editing machinery that can be crossed with incompatible lines can be created to generate edits in wide crosses. See FIG. 4. The wide cross bridge intermediate can be used in in situ embryo rescue to recover a stable wide cross with one or more genome edits. See FIG. 5. This would be particularly useful when transformation is limited to a particular genotype.

The specific examples below are to be construed as merely illustrative, and not limitative of the remainder of the disclosure in any way whatsoever. Without further elaboration, it is believed that one skilled in the art can, based on the description herein, utilize the present disclosure to its fullest extent. All publications cited herein are incorporated by reference herein in their entirety.

### Example 1: Generating Transgenic Bridge Intermediates

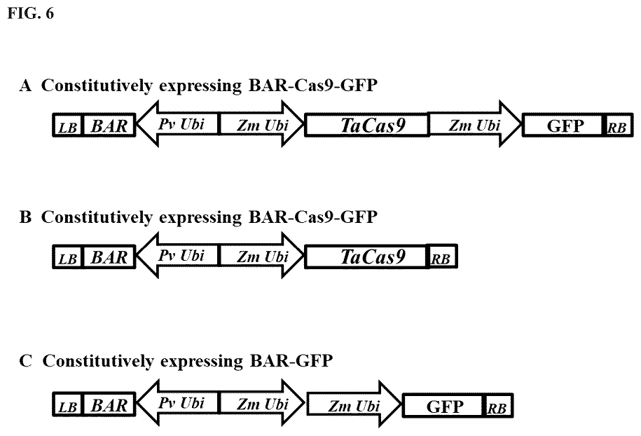

Several independent transgenic lines were made in Sorghum bicolor for each of three constructs pNG111, pNG109 and pNG108. See FIG. 6. The pNG111 vector (FIG. 6, panel A) contained a cassette designed for constitutively expressing BAR, Cas9 and GFP. The pNG109 vector (FIG. 6, panel B) contained a cassette designed for constitutively expressing BAR-Cas9. The pNG109 vector (FIG. 6, panel C) contained a cassette designed for constitutively expressing the BAR and GFP genes. Three transgenic events carrying pNG 111 (lines 3, 5, 6) were defined as single gene cassette copy inserts without rearrangements and strongly constitutively expressing the GFP gene.

A routine and high through-put method for Sorghum bicolor cv BTx430 transformation was pre-tested and used to introduce molecular constructs designed to test expression of transgenes. A sorghum transformation sequence began with immature embryos to generate transgenic embryogenic callus. Plants were grown under standard greenhouse condition until flowering. Self-pollinating Sorghum plants were bagged with a pollination bag as soon as the flower inflorescence has fully emerged. Standard and altruistic methods for generating transgenic Sorghum were used to create lines with integrated transgenes. See, Nelson-Vasilchik et al., In Vitro Cell.Dev.Biol.-Plant 58, 331-342 (2022); Kausch et al., Maize tissue culture, transformation, and genome editing. Ed. David Songstad, In Vitro Cell & Devel Biol Plant 2021. Plant Springer publ.; and Kausch et al., 2021, Maize Transformation: History, Progress and Perspectives, Molecular Breeding 41, 38, Springer publication.

Embryos were between 1.4 mm and 1.6 mm when used for transformation (at about 13-18 days past anthesis of the terminal flower). Embryos were monitored and inspected prior to harvest to ensure the correct size. The size of the embryo is the most important factor in staging of the transformation experiment. If the embryo is too small it will not survive transfection, and if it is too large it cannot be removed easily without breaking. Broken or damaged embryos will not produce embryogenic callus. Previous generation of hundreds of transgenic sorghum plants and evaluation of their T1 and T2 progeny showed efficacy of the transformation protocol.

Approximately 20-30 independent randomly inserted events per construct were created and regenerated to 3-5 plantlets per clone and grown to maturity in the TO generation. TO plants with reporter genes and sterility constructs were analyzed for their phenotypes in the TO generation, whereas all fertile transgenics, including reporter constructs, were backcrossed to wild type plants to recover T1 plants and seeded for further use in wide cross recovery and analysis. Molecular analysis was by routine PCR, qPCR for copy number determination and standard Southern blot analysis. In addition, a genomics platform has been established to follow transgene introgression in subsequent crosses. Analysis of the transgenic outcomes included both phenotypic and molecular investigations. First, reporter constructs (GFP) driven by the same promoters were analyzed in TO and T1 plants using microscopy to verify tissue specificity and the absence of ectopic expression. Verification of intact inserts was conducted by Southern blot analysis, RT-PCR, and sequencing. Concurrently, analysis of editing experiments described herein was also by florescence microscopy, X-ray microscopy and confocal microscopy.

One of the transgenic lines (pNG111 line 5) was selected as an example. T1 seeds were surface sterilized and germinated overnight in PlantCons or on moistened Whatman filter paper at 25° C. in darkness. The epicotyl and the hypocotyl emerged within 10 to 16 hours, and were scored for GFP positive expression using fluorescence microscopy.

The GFP gene was physically and genetically linked to the Cas9 gene in the cassette and served as a visual marker. The GFP was expressed strongly in the embryos of surface sterilized imbibed seeds in water and in Kausch_ngn2_Sorghum media. Approximately 10-24 hours after imbibition, the imbibed seeds were aseptically dissected through the embryo bilaterally into two halves. Dissection was accomplished using a curved scalpel blade in a sterile petri dish in a laminar flow hood, on sterile filter paper moistens with liquid Kausch_sorghum_rgn2 medium. Alternatively, tungsten surgical blades or a Vibratome was used. After surgical dissection, the halves were placed onto solid Kausch_sorghum_rgn2 medium with the cut surface up. Alternatively, the halves were placed beneath a cover glass with liquid Kausch_sorghum_rgn2 medium [MS salts, Myo-inositol, MS vitamins, 20 g/L sucrose, (+/−antioxidant mix), pH 5.8], and placed onto solid R2 medium with the cut surface up. The liquid kausch_sorghum_rgn2 medium was solidified using Phytagel to obtain a solid medium.

After dissection, the halves were imaged throughout growth and regeneration to a whole plant over 78 hours and compared with wild type controls. The embryos were imaged using a Leica m165FC Stereo microscope fitted with excitation illumination LEDs and GFP and BFP filters. Embryos were imaged using Brightfield light microscopy and the corresponding image with GFP filter in place. Embryos were incubated for 6 to 78 hours at 28° C. in darkness, observed under Brightfield light microscopy and with the GFP filter in place and used to analyze editing events. GFP was strongly expressed throughout the entire embryo, throughout both the epicotyl and hypocotyl throughout development. Growth of both the epicotyl and hypocotyl was significant (about 100 μm/hour), strongly constitutively GFP, and observable in real time. These seeds were used for in vivo genome editing, presenting the target cells in the shoot apical meristem, and were capable of growth and regeneration to a whole plant.

Example 2: Delivery of gRNAs Carried by Single-Walled Carbon Nanotubes to Epicotyl

To test the genome editing method described herein, gRNAs were designed to silence GFP expression targeting three loci and delivered by single-walled carbon nanotubes (SWNTs). GFP-minus cells appeared darker than their non-edited, untreated controls or wild type cells. The results of editing the target in GFP were observable in real time as the shoot was growing under the microscope.

Delivery experiments used a Master Mix which included independent samples of each SWNT preparations to each of the RNA guides described as follows: (1) sgSWNT 1: 70-80 mg/L of gRNA1 in an RNAase-free NaCl buffer; (2) sgSWNT 2: 70-80 mg/L of gRNA2 in an RNAase-free NaCl buffer; and (3) sgSWNT 3: 70-80 mg/L of gRNA3 in an RNAase-free NaCl buffer.

TABLE 1
Guide sequences targeting
sites in the GFP gene.

| | gRNA | | | |
|---|---|---|---|---|
| | position | | On- | Off- |
| | score/ | | target | target |
| Guide sequence | cut site | PAM | score | score |
| CTGGTCACCACCCTGACCTA | 180+ | CGG | 70 | N/A |
| gRNA1 (SEQ ID NO: 1) | | | | |
| ACCATCTTCTTCAAGGACGA | 368+ | CGG | 100 | N/A |
| gRNA2 (SEQ ID NO: 2) | | | | |
| AAAGGAGAAGAACTTTTCAC | 86+ | TGG | 85 | N/A |
| gRNA3 (SEQ ID NO: 3) | | | | |

5 to 100 seeds of BTx430 pNG111 event 5 were surface sterilized in two steps: (1) add 70% ethanol (EtOH) at room temperature to a 50 ml disposable capped tube (Corning™ or equivalent) containing the seeds and agitate for 2 minutes, decant into a beaker and rinse the seeds three times each with 50 ml of sterile (autoclaved) deionized water; (2) add 35 ml 50% sodium hypochlorite and two drops of Tween 20™ and agitate for 25 minutes, decant into a beaker and rinse the seeds five times each with 50 ml of sterile (autoclaved) deionized water. The sterilized seeds were transferred to sterile petri dishes (25/plate) with two pieces of Whatman™ filter paper moistened with sterile water and imbibed for 9-10 hours at 25° C. in darkness and scored for germination and GFP expression. Ideally, samples of germinated seeds were selected when the radicle has just emerged, and before or just after the emergence of the epicotyl. Germination is not synchronous in a sample of seeds hence development and rate of growth varied between seeds.

In the following described Delivery Experiments, unless specified otherwise, 5 to 100 seeds of BTx430 pNG111 event 5 were surface sterilized as described above. Selected aseptically grown germinated seeds were then surgically bisected in a longitudinal section through the median of the shoot apex in the epicotyl. A flamed scalpel blade could be used to make freehand sections. Preferred were tungsten surgical blades. Half-seed bisected samples were subjected to sgSWNTs treatment applications and grown to whole plants on solid Kausch_sorghum_rgn2 [MS salts, Myo-inositol, MS vitamins, 20 g/L sucrose, (+/−antioxidant mix) pH 5.8 with Phytagel @ 3-5 gm/L]. Each concentration was applied to 8 to 10 half seeds per experiment.

In some experiments, freehand sections were also prepared as approximately 150 μm thick sections through the shoot apical meristem of the epicotyl. In other examples, the 150 μm thick section of the plumule itself was isolated and used in sgSWNTs treatment applications and grown to whole plants on solid Kausch_sorghum_rgn2.

Delivery Experiment 1.0 used a dilution series of sgSWNTs 1, 2, and 3 prepared in the SWNT RNAase-free NaCl buffer comprising concentrations of 0.0 mg/L, 7.0 mg/L, 14.0 mg/L, 28.0 mg/L and 35.0 mg/L of the sgSWNT Master Mix containing sgSWNTs 1, 2, and 3 equivalently combined. Each concentration of this Master mix was applied directly to 8 to 10 half seeds per experiment. Bisected and sgSWNTs treated half seeds were either placed beneath a cover slip and observed for growth using stereoscope Brightfield and GFP imaging or allowed to grow through to plantlet regeneration on solid Kausch_sorghum_rgn2.

Delivery experiment 2.0 used 8 to 10 half seeds per experiment placed in a sterile Eppendorf tube containing sgSWNTs 1, 2, and 3 Master Mix (38 mg/L) containing sgSWNTs 1, 2, and 3 equivalently combined. Each preparation in this experiment was subjected to vacuum infiltration for 10 min. The bisected half seeds vacuum infiltrated with the sgSWNTs Master Mix were either placed beneath a cover slip and observed for growth using stereoscope Brightfield and GFP imaging or allowed to grow through to plantlet regeneration on solid Kausch_sorghum_rgn2.

In Delivery experiment 3.0 used, 8 to 10 half seeds per experiment were placed in a sterile Eppendorf tube containing OSM Medium (kausch_sorghum_rgn2; 0.4 M Mannitol). Following a 30-minute osmotic pretreatment, OSM was removed and replaced with sgSWNTs Master Mix (38 mg/L) containing sgSWNTs 1, 2, and 3 equivalently combined. The bisected half seeds with the sgSWNTs Master Mix were vacuum infiltrated for 10 min. The osmotically pretreated bisected half seeds and vacuum infiltrated with sgSWNTs Master Mix were either placed beneath a cover slip and observed for growth using stereoscope Brightfield and GFP imaging or allowed to grow through to plantlet regeneration on solid Kausch_sorghum_rgn2.

Delivery experiment 4.0 used 8 to 10 half seeds per experiment directly flooded with sgSWNTs Master Mix and were either placed beneath a cover slip and observed for growth using stereoscope Brightfield and GFP imaging or allowed to grow through to plantlet regeneration on solid Kausch_sorghum_rgn2.

In Delivery Experiment 5.0, 8 to 10 half seeds per experiment were directly flooded with sgSWNTs Master Mix (38 mg/L) containing sgSWNTs 1, 2, and 3 equivalently combined, and then subjected to microprojectile bombardment through the layer of applied sgSWNTs with untreated (naked) 0.6 μm gold particles. Bombarded samples were then either placed beneath a cover slip and observed for growth using stereoscope Brightfield and GFP imaging or allowed to grow through to plantlet regeneration on solid Kausch_sorghum_rgn2.

Delivery experiment 6.0 used 8 to 10 half seeds per experiment subjected to a cold retreatment at 4° C. in darkness for 12 hours. The seeds were transferred to 25° C. and directly flooded with sgSWNTs Master Mix (38 mg/1) containing sgSWNTs 1, 2, and 3. The seeds were then either placed beneath a cover slip and observed for growth using stereoscope Brightfield and GFP imaging or allowed to grow through to plantlet regeneration on solid Kausch_sorghum_rgn2.

Delivery Experiment 10.0 used 8 to 10 half seeds per experiment subjected to a two-pulse treatment of osmotic pretreatment followed by vacuumed infiltration with sgSWNTs. The seeds were subjected to a first pulse osmotic pretreatment [OSM, 0.4 M Mannitol, 30 min; sgSWNTs 1,2,3 (38 mg/L), 30 min; rinse in Kausch_mrng2; incubate overnight 25° C.], a second pulse osmotic pretreatment [OSM, 0.4 M Mannitol, 30 min.; sgSWNTs 1,2,3 (38 mg/1), 30 min.], and then vacuum infiltrated for 10 minutes. Seed samples were then either placed beneath a cover slip and observed for growth using stereoscope Brightfield and GFP imaging or allowed to grow through to plantlet regeneration on solid Kausch_sorghum_rgn2.

In Delivery Experiment 11.0, 8 to 10 half seeds per experiment were subjected to a cold pretreatment at 4° C. in darkness for 24 hours. The seeds were transferred to 25° C. and directly flooded with sgSWNTs Master Mix 1,2,3 (38 mg/L). The seeds were then either placed beneath a cover slip and observed for growth using stereoscope Brightfield and GFP imaging or allowed to grow through to plantlet regeneration on solid Kausch_sorghum_rgn2.

Delivery Experiment 12.0 used 8 to 10 half seeds per experiment subjected to microprojectile bombardment with sgSWNTs incubated with 0.6 μm gold particles. Samples bombarded with the sgSWNTs particles were then either placed beneath a cover slip and observed for growth using stereoscope Brightfield and GFP imaging or allowed to grow through to plantlet regeneration on solid Kausch_sorghum_rgn2.

Example 3: Delivery of gRNAs Carried by Non-Integrating FoMV to Epicotyl

The Sorghum bmr6 gene encodes an orthologous cinnamyl alcohol dehydrogenases (CAD) in the lignin synthesis pathway. The major components of lignin are monolignols, which belong to the class of phenolic hydroxycinnamyl alcohol monomers, such as p-coumaryl, coniferyl, and syringyl alcohols. Lignin impedes the saccharification of cellulose, probably by reducing the accessibility of the saccharification enzymes to the cellulose microfibrils. Consequently, breeding sorghum cultivars that accumulate less lignin is an important strategy for improving the use of these materials in biofuel production.

The sorghum brown midrib mutant 6 (bmr6) has a reduced lignin content in the cell walls and vascular tissues, which could potentially be advantageous for cellulosic biofuel production and higher forage digestibility. Brown midrib mutants identified by the reddish-brown color of their leaf midrib that is initially usually visible at the four- to six-leaf stage.

sgRNAs have been demonstrated to be expressed from the duplicated promoter, mediating edits in the N. benthamiana PDS gene, the S. viridis carbonic anhydrase 2 gene, and the Zea mays HKT1 gene encoding a potassium transporter in monocots using the foxtail millet mosaic virus FoMV.

Guide RNAs were designed to target five sites in the bmr6 gene. See Table 2. Vectors were prepared in FoMV.

TABLE 2
Guide sequences targeting sites
in the bmr6 gene in Sorghum.

| | |
|---|---|
| gRNA-1 (SEQ ID NO: 4) | GGTACTTTGAAGCCCCGAGG |
| gRNA-2 (SEQ ID NO: 5) | ACGATGTCTACACTGACGGC |
| gRNA-3 (SEQ ID NO: 6) | CGGGGCTTCAAAGTACCCTA |
| gRNA-4 (SEQ ID NO: 7) | CCACCTTCCTCTCGGACGCC |
| gRNA-5 (SEQ ID NO: 8) | GCGGCCTGGAGAGGCCGAGA |

FoMV sap was prepared from Agroinfiltration of FoMV infected N. benthamiana to generate preparations FoMV bmr6 1, 2, 3, 4, and 5 each with guides 1-FoMV-bmr6-gRNA-1, 2-FoMV-bmr6-gRNA-2, 3-FoMV-bmr6-gRNA-3, 4-FoMV-bmr6-gRNA-4, and 5-FoMV-bmr6-gRNA-5, respectively. Sap was prepared directly from the crushed leaves of the Agroinfected N. benthamiana.

A Master Mix was made containing FoMV bmr6 1, 2, 3, 4, and 5 with guides 1-FoMV-bmr6-gRNA-1, 2-FoMV-bmr6-gRNA-2, 3-FoMV-bmr6-gRNA-3, 4-FoMV-bmr6-gRNA-4, and 5-FoMV-bmr6-gRNA-5, respectively, equivalently combined. Each treatment in the example used this Master Mix, or in various combinations of the individual FoMVs, as well as independently for each the FoMV bmr6 1, 2, 3, 4, and 5 with guides 1-FoMV-bmr6-gRNA-1, 2-FoMV-bmr6-gRNA-2, 3-FoMV-bmr6-gRNA-3, 4-FoMV-bmr6-gRNA-4 and 5-FoMV-bmr6-gRNA-5, respectively.

Seeds (5-100) of BTx430 pNG111 event 5 were surface sterilized in two steps: (1) add 70% ethanol (EtOH) at room temperature to a 50 ml disposable capped tube (Corning™ or equivalent) containing the seeds and agitate for 2 minutes, decant into a beaker and rinse the seeds three times each with 50 ml of sterile (autoclaved) deionized water; (2) add 35 ml 50% sodium hypochlorite and two drops of Tween 20™ and agitate for 25 minutes, decant into a beaker and rinse the seeds five times each with 50 ml of sterile (autoclaved) deionized water. The sterilized seeds were transferred to sterile petri dishes (25/plate) with two pieces of Whatman™ filter paper moistened with sterile water and imbibed for 9-10 hours at 25° C. in darkness and scored for germination and GFP expression. Ideally, samples of germinated seeds were selected when the radicle has just emerged, and before or just after the emergence of the epicotyl. Note that germination is not synchronous in a sample of seeds hence development and rate of growth varies between seeds.

Unless otherwise specified, seeds (5-100) of BTx430 pNG111 event 5 were surface sterilized as described. Selected aseptically grown germinated seeds were surgically bisected in a longitudinal section through the median of the shoot apex in the epicotyl. A flamed scalpel blade could be used to make freehand sections. Preferred were tungsten surgical blades. Half-seed bisected samples were subjected to FoMV bmr6 1, 2, 3, 4, and 5 treatments and grown to whole plants on solid Kausch_sorghum_rgn2 (MS salts, Myo-inositol, MS vitamins, 20 g/L sucrose, (+/−antioxidant mix) pH 5.8 with Phytagel @ 3-5 gm/L). Each concentration was applied to 8 to 10 half seeds per experiment.

In Delivery Experiment 13.0, treatments were applied directly to 8-10 half seeds per experiment. Each preparation in this experiment was subjected to treatment with the Master Mix of FoMV bmr6 1,2,3,4, and 5, or each guide independently with vacuum infiltration for 10 minutes. The bisected half seeds vacuum infiltrated with the treatments were either placed beneath a cover slip and observed for growth using stereoscope Brightfield and GFP imaging or allowed to grow through to plantlet regeneration on solid Kausch_sorghum_rgn2.

Treatments were applied directly to 8-10 half seeds per experiment in Delivery Experiment 14.0. Each preparation in this study was subjected to osmotic pretreatment with OSM, 0.4 M Mannitol, for 30 minutes and then treatment with the Master Mix of FoMV bmr6 1, 2, 3, 4, and 5 with vacuum infiltration for 10 minutes. Bisected seeds were independently treated for each of guides 1-FoMV-bmr6-gRNA-1, 2-FoMV-bmr6-gRNA-2, 3-FoMV-bmr6-gRNA-3, 4-FoMV-bmr6-gRNA-4 and 5-FoMV-bmr6-gRNA-5. The bisected half seeds vacuum infiltrated with the treatments were either placed beneath a cover slip and observed for growth using stereoscope Brightfield and GFP imaging or allowed to grow through to plantlet regeneration on solid Kausch_sorghum_rgn2.

In Delivery Experiment 15.0, treatments were applied directly to 8-10 half seeds per experiment. Each preparation in this experiment was subjected to direct incubation with the Master Mix of FoMV bmr6 1, 2, 3, 4, and 5 with vacuum infiltration for 10 minutes. Bisected seeds were independently treated for each the FoMV bmr6 1,2,3,4,5 with guides 1-FoMV-bmr6-gRNA-1, 2-FoMV-bmr6-gRNA-2, 3-FoMV-bmr6-gRNA-3, 4-FoMV-bmr6-gRNA-4 and 5-FoMV-bmr6-gRNA-5. The bisected half seeds vacuum infiltrated with the treatments were either placed beneath a cover slip and observed for growth using stereoscope Brightfield and GFP imaging or allowed to grow through to plantlet regeneration on solid Kausch_sorghum_rgn2.

Delivery Experiment 16.0 used direct incubation with FoMV bmr6 1, 2, 3, 4, and 5 followed by microprojectile bombardment with untreated gold particles. Each preparation in this study was subjected to direct incubation with a Master Mix of all 5 FoMV bmr6 guides or each guide separately. After application of the virus sap, the seeds were subjected to microprojectile bombardment with untreated (naked) 0.6 μm gold particles through the layer of applied FoMVs. The bisected half seeds bombarded through the treatments were either placed beneath a cover slip and observed for growth using stereoscope Brightfield and GFP imaging or allowed to grow through to plantlet regeneration on solid Kausch_sorghum_rgn2.

In Delivery Experiment 17.0, 8 to 10 half seeds per experiment were subjected to cold retreatment at 4° C. in darkness for 12 hours. The seeds were transferred to 25° C. and directly flooded with Master Mix FoMVs 1, 2, 3, 4, and 5. The seeds were then either placed beneath a cover slip and observed for growth using stereoscope Brightfield and GFP imaging or allowed to grow through to plantlet regeneration on solid Kausch_sorghum_rgn2.

Delivery Experiment 18.0 used two pulses of FoMV treatments. In the first pulse, bisected seeds were subjected to osmotic pretreatment (OSM, 0.4 M Mannitol) for 30 minutes, Master Mix FoMVs 1, 2, 3, 4, and 5 for 30 minutes, followed by vacuum infiltration for 10 minutes. The treated seeds were rinsed briefly with Kausch_mrng2 and incubated overnight at 25° C. The second pulse was a repeat of the first pulse.

In Delivery Experiment 19.0, 8 to 10 half seeds per experiment were subjected to cold treatment at 4° C. in darkness for 24 hours. The seeds were then transferred to 25° C. and directedly flooded with Master Mix FoMVs 1, 2, 3, 4, and 5. The seeds were then either placed beneath a cover slip and observed for growth using stereoscope Brightfield and GFP imaging or allowed to grow through to plantlet regeneration on solid Kausch_sorghum_rgn2.

### Example 4: Results of Delivery Experiments

The results of the above-described Delivery Experiments were analyzed by various methods including a range of microscopy techniques for phenotypic analysis and sequencing through the target sites in treated plants and controls. The experiments targeting the GFP sequence were analyzed for GFP expression using a Leica M165 FC with apochromatic corrected lenes stereo microscope with 16.5:1 zoom optics, resolving structures down to 1.1 micron to provide detailed high resolution fluorescent imaging for GFP analysis. A time course sequence of development of the seed sections was first observed on control sections without any guide RNA delivery. Specimens were analyzed from time zero through the first 72 hours of all experimental treatments in the above-described experiments using the Leica M165 FC high resolution stereo microscopy.

In one representative example of a developmental time course, seeds were surface sterilized and incubated for 21 hours and surgically dissected as described herein. Epicotyl sections of the seeds were observed and imaged during development under a glass coverslip in Kausch_sorghum_rgn2 at approximately 4-hour intervals. At time zero, the epicotyl has not yet emerged from the seed coat. Strong GFP expression was observed through all cells and tissues of the germinating embryo, including the cells and tissues of the scutellum, and the embryonic axis, including the coleoptile, the plumule, the shoot apical meristem (SAM), the mesocotyl, the radicle, the root apical meristem (RAM), and the coleorhiza. Weak fluoresce was observed throughout the aleurone, the basal endosperm transfer layer (BETL), embryo surrounding region (ESR) and starchy endosperm (SE). The pericarp was strongly auto fluorescent. Variation in intensity of GFP fluorescence was consistent with vacuolation of cells in the various tissues of the embryo. Cytoplasmicly dense cells in the SAM and RAM were most strongly GFP florescent. Cells walls were resolved and appear as dark outlines for individual cells.

The time course showed development of the dissect embryo to a seedling stage from time zero to 72 hours post emergence from the seed coat and imaged at 4-hour intervals. After 4 hours, the epicotyl has emerged approximately 1.0-1.5 mm from the seed coat. After 8 hours, the epicotyl has emerged approximately 1.5-2.5 mm from the seed coat with significant growth in the mesocotyl and shoot elongation. Bubbles formed under the glass coverslip indicated ongoing cellular respiration during germination. After 12 hours, the epicotyl has emerged approximately 2.0-3.5 mm from the seed coat. After 16 hours, the epicotyl has emerged approximately 3.0-5.0 mm from the seed coat; dark cells were observed at the cut surface of developing leaves and cells of the developing shoot and the scutellum remained strongly fluorescent. After 20 hours, the epicotyl has emerged approximately 4.0-7.0 mm from the seed coat and was recognizable as a small shoot by 24 hours post emergence with strong florescence throughout the shoot and the root. After 72 hours, the young shoot was a small plant which was recovered to a fertile plant with constitutive GFP expression.

GFP expression was then observed after Delivery Experiments as described above throughout development or the first 72 hours using the Leica THUNDER Imager Model Organism System with 1× Plan APO objective, zoom factor 11:1, 18 z-slice stack, circa 300 μm z-depth. Silencing of GFP expression was consistently observed across all treatments. Gradual decrease in GFP fluorescence was observed during the first 24 hours, resulting in a darkened shot and root. The scutellum remained strongly florescent. Subsequent analysis using the Leica FLIM Stellaris 8 Falcon Micah Model inverted microscope showed florescence consistent with GFP silencing in the root and shoot tissues.

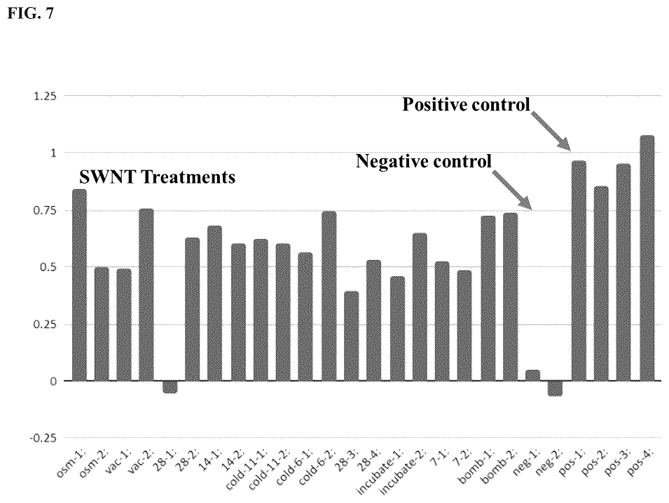

Shoots from treated epicotyls were regenerated and grown in aseptic culture and then transferred to soil. After the first five leaves had emerged and expanded, a 2-centimeter apical section of the fifth leaf was sampled and analyzed using the Leica Stellaris 8 FALCON System confocal microscope. Phasor analysis with median filter 21 the ratio cursor for two component analysis revealed a consistent and significant reduction in GFP florescence compared with the positive (untreated) controls and calibrated against the negative wild-type control tissues. See FIG. 7. Some samples showed entire elimination of GFP fluorescence at levels equal to the negative control (see FIG. 7, sample 28.1).

In the experiments described above using the non-integrating virus FoMV carrying the guide RNAs to target sequences in the bmr6 gene in Sorghum, the brown midrib phenotype was observed in shoots regenerated from samples treated with FoMV sap preparations carrying gRNAs and multiplexed gRNAs.

Genomic DNA samples were taken from leaves of fully grown mature plants from all treatments in the above-described experiments. DNA sequence data showed targeted deletion of the GFP gene at the sites specified by the guide sequences. Deletions defined by the targeted gRNA1 and gRNA2 confirmed specificity of editing the target GFP sequence and that the guide RNAs were specific when multiplexed.

Results from the above-described delivery experiments showed that direct genome editing was achieved in the epicotyl of the seed sections. The genome editing platform was shown to be compatible with multiple delivery methods including carbon nanotubes (CNTs or SWNTs) and non-integrating virus, such as FoMV. The evidence from various levels of microscopy showed the effects of disrupting GFP expression with gRNAs delivered by SWNTs, including quantitative confocal microscopy evidencing directed genome editing in all treatments. Sequence evidence showed that the disruption was due to the targeted sequences being affected and correctly edited.

The above-described experiments showed that two independent genes (GFP and BRM6) were successfully targeted and edited to produce phenotypes in plants and that guide RNAs can be multiplexed in the methods described herein.

What is claimed is:

1. A method of genome editing in an explant, comprising:
obtaining a seed of a bridge transgenic plant having a stably integrated Cas expression cassette containing a nucleic acid encoding a Cas protein;
treating the seed to obtain a germinated seed, wherein the germinated seed contains a viable epicotyl and the Cas protein is expressed in the epicotyl; and
carrying out genome editing in the germinated seed.

2. The method of claim 1, further comprising:
prior to the genome editing step, cutting the germinated seed to produce a seed section in which a portion of shoot apical meristem is exposed or proximate to an exposed surface of the seed section; and
applying a genome editing reagent directly in vivo to the portion of shoot apical meristem or the exposed surface of the seed section under conditions allowing delivery of the genome editing reagent into a cell of the shoot apical meristem, wherein the cell expresses the Cas protein, whereby genome editing occurs in the cell to produce at least one genomic edit.

3. The method of claim 1, wherein the bridge transgenic plant is a monocotyledonous, dicotyledonous, monoploid, diploid, triploid, or polyploid plant.

4. The method of claim 2, wherein the seed section is produced by cutting the germinated seed longitudinally.

5. The method of claim 2, wherein the seed section is produced by isolating a section of the germinated seed containing the shoot apical meristem.

6. The method of claim 2, wherein the genome editing reagent includes a guide RNA (gRNA).

7. The method of claim 6, wherein the genome editing reagent contains two or more gRNAs for making at least two different genome edits, whereby genome editing of at least two loci occurs in the cell of the shoot apical meristem, and wherein at least one of the genome edits confers a selective advantage under a selective agent or condition.

8. The method of claim 7, wherein the selective advantage is resistance or tolerance to a condition or toxic agent.

9. The method of claim 7, further comprising applying the selective agent or condition to the seed section before, after or at the same time that the genome editing reagent is applied, whereby conferring the selective advantage to the cell containing the at least two genome edits.

10. The method of claim 2, wherein the applying step includes substantially covering the portion of shoot apical meristem or the exposed surface with a composition containing the genome editing reagent.

11. The method of claim 10, further comprising subjecting the seed section to (i) vacuum filtration or (ii) microprojectile bombardment.

12. The method of claim 10, further comprising subjecting the seed section to one or both of a cold pretreatment and osmotic pretreatment prior to or at the same time with the applying step.

13. The method of claim 2, wherein the genome editing reagent includes a vector or delivery vehicle carrying a nucleic acid.

14. The method of claim 13, wherein the vector is a viral vector or the delivery vehicle is a carbon nanotube.

15. A method of producing a genome-edited plant, comprising:
obtaining a seed of a bridge transgenic plant having a stably integrated Cas expression cassette containing a nucleic acid encoding a Cas protein;
treating the seed to obtain a germinated seed, wherein the germinated seed contains a viable epicotyl and the Cas protein is expressed in the epicotyl;
surface sterilizing the germinated seed;
cutting the sterilized seed to produce a seed section in which a portion of shoot apical meristem is present and exposed or proximate to an exposed surface;
applying a genome editing reagent directly in vivo to the exposed surface or the portion of shoot apical meristem of the seed section under conditions allowing delivery of the genome editing reagent into a cell of the shoot apical meristem, wherein the cell expresses the Cas protein, whereby genome editing occurs in the cell to produce at least one genome edit;
growing the seed section under conditions allowing whole plant regeneration, whereby an F1 genome-edited plant is produced.

16. The method of claim 15, wherein the bridge transgenic plant is a monocotyledonous, dicotyledonous, monoploid, diploid, triploid or polyploid plant.

17. The method of claim 15, wherein the genome editing reagent includes a guide RNA (gRNA).

18. The method of claim 17, wherein the genome editing reagent contains two or more gRNAs for making at least two different genome edits, whereby genome editing of at least two loci occurs in the cell of the shoot apical meristem, and wherein at least one of the genome edits confers a selective advantage under a selective agent or condition and at least one of the genome edits confers a target trait, and wherein the F1 genome-edited plant contains both genome edits.

19. The method of claim 18, wherein the selective advantage is resistance or tolerance to a condition or toxic agent.

20. The method of claim 18, further comprising applying the selective agent or condition to the seed section before, after or at the same time that the genome editing reagent is applied, or before or during the growing step, whereby conferring the selective advantage to the cell containing the at least two genome edits.

21. The method of claim 15, further comprising producing from the F1 genome-edited plant a hybrid embryo, seed or plant containing the genome edit conferring the target trait in which the Cas expression cassette is removed by segregation in subsequent generations.

22. The method of claim 21, wherein the hybrid embryo, seed or plant is an F1BC1 or F1BC2 hybrid embryo, seed or plant produced by backcrossing the F1 genome-edited plant to a plant germplasm having the same or closely syntenic genotype as the F1 genome-edited plant but lacking the Cas expression cassette and the at least one genome edit.

23. The method of claim 21, wherein an offspring containing the genome edit conferring the target trait is segregated away from the Cas expression cassette to produce a non-GMO, genome-edited intact fertile plant.

24. The method of claim 18, further comprising producing from the F1 genome-edited plant a hybrid embryo, seed or plant containing the genome edit conferring the target trait in which the Cas expression cassette and the genome edit conferring the selective advantage are both removed by segregation in subsequent generations.

25. The method of claim 23, wherein the hybrid embryo, seed or plant is an F1BC1 or F1BC2 hybrid embryo, seed or plant produced by backcrossing the F1 genome-edited plant to a plant germplasm having the same or closely syntenic genotype as the F1 genome-edited plant but lacking the Cas expression cassette and the at least one genome edit.

26. The method of claim 24, wherein an offspring containing the genome edit conferring the target trait is segregated away from the Cas expression cassette and the genome edit conferring the selective advantage to produce a non-GMO, genome-edited intact fertile plant.

27. The method of claim 15, further comprising using the F1 genome-edited plant in a wide cross to a plant germplasm having a distantly syntenic genotype as the F1 genome-edited plant lacking the Cas expression cassette and the genome edit conferring the selective advantage to produce a bridge intermediate F1BC1 hybrid embryo, seed or plant.

28. The method of claim 27, wherein an offspring containing the genome edit conferring the target trait is segregated away from the Cas expression cassette and the genome edit conferring the selective advantage to produce a non-GMO, genome-edited intact fertile plant.

29. The method of claim 27, wherein an F2 hybrid embryo, seed or plant is recovered to produce a fertile embryo, seed or hybrid plant using in situ embryo rescue.


SEQUENCE LISTING (8 sequences)

SEQ ID NO: 1 - RNA, 20 nt, synthetic construct
  source 1..20: mol_type = other RNA
  misc_feature 1..20: note = GFP gRNA1
ctggtcaccaccctgaccta

SEQ ID NO: 2 - RNA, 20 nt, synthetic construct
  source 1..20: mol_type = other RNA
  misc_feature 1..20: note = GFP gRNA2
accatcttcttcaaggacga

SEQ ID NO: 3 - RNA, 20 nt, synthetic construct
  source 1..20: mol_type = other RNA
  misc_feature 1..20: note = GFP gRNA3
aaaggagaagaacttttcac

SEQ ID NO: 4 - RNA, 20 nt, synthetic construct
  source 1..20: mol_type = other RNA
  misc_feature 1..20: note = bmr6 gRNA-1
ggtactttgaagccccgagg

SEQ ID NO: 5 - RNA, 20 nt, synthetic construct
  source 1..20: mol_type = other RNA
  misc_feature 1..20: note = bmr6 gRNA-2
acgatgtctacactgacggc

SEQ ID NO: 6 - RNA, 20 nt, synthetic construct
  source 1..20: mol_type = other RNA
  misc_feature 1..20: note = bmr6 gRNA-3
cggggcttcaaagtacccta

SEQ ID NO: 7 - RNA, 20 nt, synthetic construct
  source 1..20: mol_type = other RNA
  misc_feature 1..20: note = bmr6 gRNA-4
ccaccttcctctcggacgcc

SEQ ID NO: 8 - RNA, 20 nt, synthetic construct
  source 1..20: mol_type = other RNA
  misc_feature 1..20: note = bmr6 gRNA-5
gcggcctggagaggccgaga